(19) United States
(12) Patent Application Publication
(10) Pub. No.: US 2019/0009050 A1
(43) Pub. Date: Jan. 10, 2019

(54) System and Method of Manufacturing Dialysis Catheters

(71) Applicant: VascuTech Medical LLC, Oaks, PA (US)

(72) Inventor: Mahase Nardeo, Oaks, PA (US)

(73) Assignee: VascuTech Medical LLC, Oaks, PA (US)

(21) Appl. No.: 15/641,608

(22) Filed: Jul. 5, 2017

(51) Int. Cl. A61M 25/00; A61M 1/36; B29C 35/08; B29C 43/02; B29C 43/52

(52) U.S. Cl. CPC A61M 25/001; A61M 25/0068; A61M 1/3661; A61M 25/003; B29L 2031/7542; B29C 43/02; B29C 43/52; A61M 2025/0031; B29C 2035/0861; B29C 35/0805

(57) ABSTRACT

Disclosed is a system and method for splitting dual lumens using hot stamping. The system includes a die assembly having a heated die with a septum that is configured to split the dual lumen to form a split catheter by splitting the septum of the dual lumen. The die can receive a pair of mandrels therethrough, wherein the mandrels are configured to support a dual lumen loaded thereon. The dual lumen and the mandrels are pushed via a block into the die until the dual lumen passes through the septum within the die and two lumens are fully formed. The lumens are cooled with an air blast and the mandrels are removed after the lumens are cooled.

### CROSS-REFERENCE TO RELATED APPLICATIONS

This application claims the benefit of U.S. Provisional Patent Application No. 62/361,099, filed Jul. 12, 2016, which is hereby incorporated by reference in its entirety.

### FIELD OF THE INVENTION

The present invention relates generally to medical devices, namely, catheters. More particularly, the present invention is directed to a system and method of manufacturing dialysis catheters via a heat-forming die.

### BACKGROUND OF THE INVENTION

Split tip catheters are used widely in dialysis. As the name implies, split tip catheters comprise dual lumens having a split tip. Various methods of manufacturing split tip catheters are known in the art. Primarily, these methods comprise the steps of splitting dual lumen tubes in the middle to form two tips, bonding two lumens (having D-shaped cross section) together to form a catheter while leaving a desired split length, skiving one D-shaped lumen off a dual lumen tubes and fusing another D-shaped lumen onto the skived tubes, and using a sleeve to bond or hold two lumens together and leaving the desired length separated.

The existing methods, however, are limiting in that they result in catheters having a larger profile or a larger cross-sectional diameter because splitting septum of a dual lumen is a very challenging process. Thus, catheters that are produced using the existing methods can be uncomfortable for patients and not desirable to use. In this regard, the invention described herein addresses this problem.

### SUMMARY OF THE INVENTION

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as a prelude to the more detailed description that is disclosed later.

In one embodiment, the present method generally comprises the steps of utilizing heated die to split dual lumen tubes, wherein the lumens are separated via a septum that is disposed in the middle of the die. The present method is advantageous in that regular dual lumen tubes without a thicker septum (i.e., a septum having a thickness that is substantially equal to the thickness of the remaining parts of the lumens) can be used. In this way, the cross-sectional diameter of the catheter that is manufactured via the present method is less than the cross-sectional diameter of the catheter that is manufactured via existing means.

### BRIEF DESCRIPTION OF THE DRAWINGS

FIGS. 1A through 1C depict cross-sectional views of split tip catheters in the prior art.

FIG. 1D shows a cross-sectional view of a split tip catheter of the present invention.

FIG. 2 shows a representative side cross-sectional view of an exemplary die assembly for forming a split tip catheter of the present invention.

FIG. 3 shows a front cross-sectional view of the die assembly.

FIG. 4 shows an exemplary block diagram of a computer system connected to the heating system of the die assembly.

FIG. 5 shows high-level exemplary method steps of the present invention.

### DETAILED DESCRIPTION OF THE INVENTION

For purposes of promoting an understanding of the present disclosure, reference is made to the embodiments illustrated in the above-referenced drawings. The following detailed description of the exemplary embodiments will make clear the arrangement, size relationships, and manner of using the components shown herein.

In the following discussion, the terms “proximal” and “distal” are used to describe the axial ends of the catheter, as well as the axial ends of various component features. The “proximal” end is used in a conventional manner to refer to the end of the catheter (or component) that is closest to the operator during use of the assembly. The “distal” end is used in a conventional manner to refer to the end of the catheter (or component) that is initially inserted into the patient, or that is closest to the patient. Additionally, those skilled in the art will appreciate that the catheter assembly described herein is suitable for multiple uses involving inflow and outflow of body fluids from a body vessel of a patient.

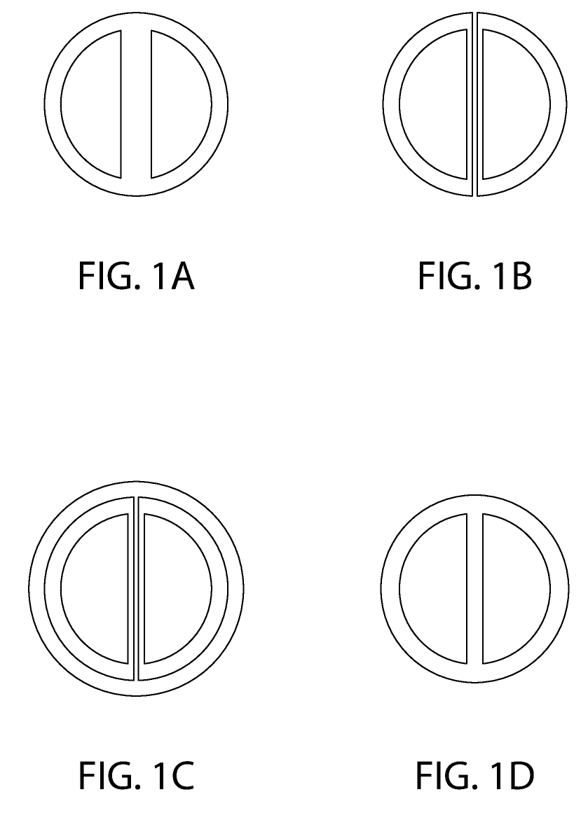

Referring now to FIGS. 1A through 1C, there are shown views of existing split tip catheters produced via existing methods. The catheters are composed of a range of polymers typically used for the construction of catheters, including silicone rubber, nylon, polyurethane, polyethylene terephthalate (PET), latex, and thermoplastic elastomers. Each of the catheters in FIGS. 1A through 1D comprises a dual lumen catheter having a venous lumen and an arterial lumen, separated via a septum, wherein each of the lumens comprises a proximal end and a distal end. The lumens are split toward the distal ends thereof so that each separate lumen comprises a D-shaped cross section. The lumens are held together toward the proximal ends thereof.

FIG. 1A shows a catheter having a thick septum for facilitating splitting method technology. Because the thickness of the septum is relatively greater than the thickness of the walls of the lumens, the lumens can be separated relatively easily. Once split, the thickness of the cut septum on each lumen is substantially equal to the thickness of the walls of the lumens. FIG. 1C shows two separate lumens that are bonded or secured together in a side-by-side configuration via a sleeve. The sleeve is a cylindrical member that fits over the two lumens. The lengths of the lumens extend beyond the length of the sleeve (i.e., distance between the proximal and distal ends of the sleeve) so that a portion of the lumens can be held together (i.e., the proximal ends) while the remaining portion of the lumens (i.e., the distal ends) can remain separated. However, the thickness of the septum as shown in FIG. 1A and the addition of the sleeve as shown in FIG. 1C increase the overall size of the cross section of the catheter. Thus, the catheter as shown in FIGS. 1A and 1C can increase discomfort to a patient during use.

FIG. 1B shows a split catheter that was split using a skiving method technology. The skiving method, however, does not result in smooth septa because the cut can be uneven and there are no methods to improve the finish once the lumens are split. Finally, FIG. 1D shows regular dual lumen tubes that can form a split tip by using a heat forming die or heat stamping. It is noted that the regular dual lumen tubes comprise a cross-sectional diameter that is less than the cross-sectional diameter of other types of catheters as shown in FIGS. 1A and 1C.

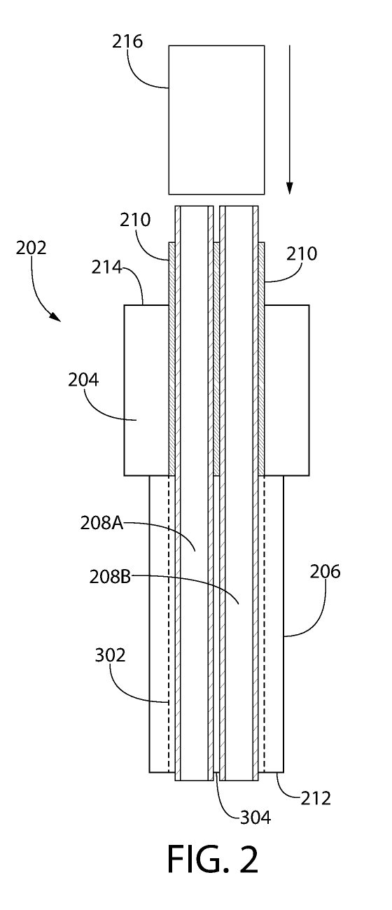

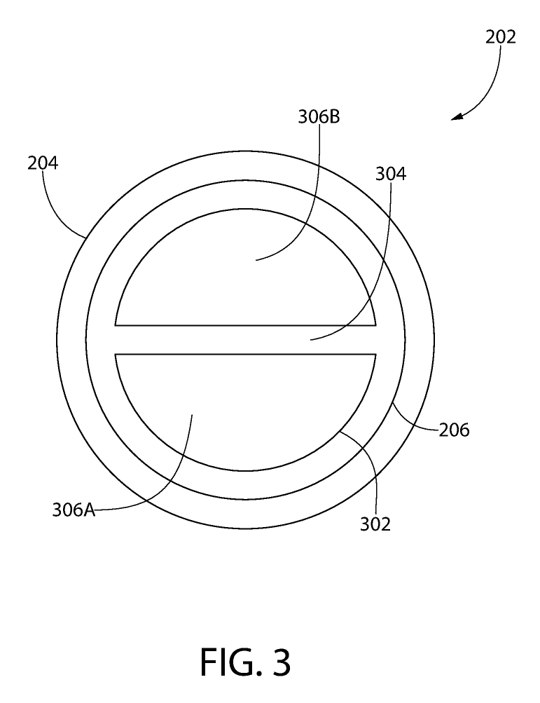

Referring now to FIGS. 2 and 3, there is shown a side cross-sectional of an exemplary die assembly 202 for manufacturing a split tip catheter, and a front cross-sectional view of the die assembly, respectively. The die assembly 202 comprises a die holder 204 having a die 206 attached thereto at a distal end thereof. The die holder 204 comprises a hollow cylindrical body having a substantially circular cross-section. Each end (i.e., the distal end and proximal end) of the die holder 204 comprises an opening that provides an access to the hollow interior thereof. Thus, the interior of the die holder 204 comprises a tunnel. The opening at the distal end of the die holder 204 is aligned with the proximal end of the die 204.

The die 206 can be composed of metal or silicone rubber and can be shaped directly or cast. The die 206 comprises a hollow cylindrical body having a circular cross section. The diameter of the cross section of the die 206 is less than the diameter of the cross section of the die holder 204. The die 206 and the die holder 204 are shaped and dimensioned so that the outer wall of the die 206 forms a complete seal around the inner wall of the die holder 204.

Similar to the die holder 204, the die 206 comprises an open distal end and an open proximal end, each end providing an access to the hollow interior of the cylindrical body to form a tunnel. The diameter of the cross section of the tunnel of the die holder 204 is substantially equal to the diameter of the cross section of the tunnel of the die 206. In this way, the tunnel of the die holder 204 and the tunnel of the die 204 are substantially unitary in structure so as to form a single tunnel. The single tunnel is configured to receive a dual lumen and a pair of mandrels therethrough.

The inner wall 302 of the die 206 comprises a smooth surface. The die 206 further comprises a septum 304 that spans across the inner wall 302 of the die 206. Thus, the septum defines a first compartment 306A and a second compartment 306B in the interior volume of the die 206. The septum is disposed at a substantial midsection of the inner wall of the die 206 so that the first compartment 306A and the second compartment 306B comprise two semicircle sections that are substantially equal in size and dimension.

A first mandrel 208A and a second mandrel 208B can be inserted through the proximal end 214 of the die holder 204 so that the first mandrel 208A is extended through the die holder 204 and the first compartment 306A of the die 206, and the second mandrel 208B is extended through the die holder 204 and the second compartment 306B of the die 206, whereby the first mandrel 208A and the second mandrel 208B are in a substantially side-by-side configuration. Thereafter, a dual lumen 210 can be loaded onto the mandrels 208A, 208B, wherein one of the lumens is loaded onto the first mandrel 208A and the other lumen is loaded onto the second mandrel 208B through the proximal end 214 of the die holder 204. It is noted that the dual lumen 210 is initially intact when loaded onto the mandrels 208A, 208B and therefore not split. Each of the lumens of the dual lumen 210 remains loaded on each respective mandrel 208A, 208B until removed to prevent the lumens from collapsing during the splitting—particularly, the heating—process.

Because the septum 304 extends through the tunnel of the die 206, the septum 304 initially blocks the dual lumen 210 from being inserted all the way through the tunnel of the die 206. Once the dual lumen 210 is loaded onto the mandrels 208A, 208B, the proximal ends of the lumen 210 and the mandrels 208A, 208B are pushed or guided all the way through the die assembly 202 toward the distal end 212 of the die 206 via a block, a press, or a guide 216 that can be integral to the die assembly 202. The block 216 is shaped and dimensioned to be inserted into the die holder 204 to ensure that the dual lumen 210 and the mandrels 208A, 208B are pushed all the way through. As the dual lumen 210 and the mandrels 208A, 208B are pushed through the die 206 and heat is applied to the dual lumen 210, the dual lumen 210 can be split.

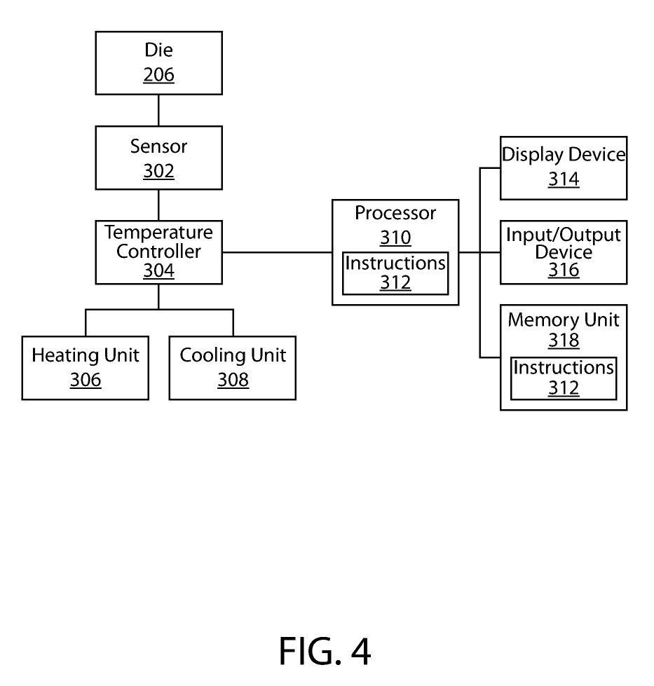

As depicted in FIG. 4, the die 206 is operatively connected to a heating unit 306 (e.g., a radio frequency heating system) for heating the die 206, for example, using radio frequency or other suitable methods. The heat from the die 206 creates a smooth finish when the dual lumen 210 and the mandrels 208A, 208B are pushed through the die 206 and the lumens become split. The temperature of the die 206 can be controlled and monitored via a temperature controller 304 that is connected to the heating unit 306, wherein the temperature controller 304 can communicate with a sensor 302 (e.g., thermometer) for detecting the temperature of the die 206.

Additionally, the temperature controller 304 can be coupled to a machine in the example form of a computer system within which instructions, for causing the machine to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a Personal Digital Assistant (PDA), cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system includes a processor 310 (e.g., a central processing unit (CPU), a graphics processing unit (GPU)) and a memory unit 318 (e.g., a main memory unit, a static memory unit), which communicate with each other via a bus. The computer system may further include a display device 314 (e.g., a liquid crystal display (LCD) or a cathode ray tube (CRT)). The computer system also includes various input/output devices 316 such as an alphanumeric input device (e.g., a keyboard), a user interface (UI) navigation device (e.g., a mouse), a disk drive unit, a signal generation device (e.g., a speaker), and a network interface device.

The disk drive unit includes a machine-readable medium on which is stored one or more sets of data structures and instructions 312 (e.g., software) embodying or utilized by any one or more of the methods or functions described herein. The instructions 312 may also reside, completely or at least partially, within the memory unit 318 and/or within the processor 310 during execution thereof by the computer system. In this regard, the memory unit 318 and the processor 310 are also considered machine-readable media.

The instructions 312 may further be transmitted or received over a computer network using a transmission medium. The instructions 312 may be transmitted using the network interface device and any one of a number of well-known transfer protocols. The term “transmission medium” shall be taken to include any intangible medium that is capable of storing, encoding, or carrying instructions for execution by the machine, and includes digital or analog communications signals or other intangible media to facilitate communication of such software.

The die 206 provides temperature feedback during operation. The temperature can be displayed, for example, on the display device 314 of the computer system. The temperature controller 304 determines whether the temperature of the die 206 is within the target temperature range. In a preferred embodiment, the target temperatures for heating the die 206 have a range between 300° F. and 350° F. It is noted, however, that the temperature can range depending on the material of the catheter.

The target temperatures can be obtained, for example, through frequency modulation using the computer system. More specifically, if the temperature controller 304 determines that the temperature of the die 206 falls outside of the target temperature range, the processor 310 can automatically instruct the heating unit 306 via the temperature controller 304 to either raise or lower the temperature. Alternatively, the heating unit 306 can be manually operated via the input/output device 316 in order to receive user input and apply heat to the die 206. In some embodiments, the processor 310 is configured to automatically determine the target temperatures for heating the die 206 depending on the material of the catheter by referring to a data sheet or a source in a database connected thereto. In this regard, it is contemplated that a user can input the type of material that the catheter is composed of in order for the processor 310 to automatically determine the appropriate temperature range.

The die 206 is further connected to a cooling unit 308 for cooling the lumens. In a preferred embodiment, the cooling unit 308 is configured to provide an air blast to cool the lumens 210. The cooling unit 308 can be operatively connected to the computer system via the temperature controller 304 for operating the cooling unit 308. The cooling unit 308 can automatically provide air blast after the lumens are split. Alternatively, the cooling unit 308 can be manually operated via the input/output device 316 of the computer system.

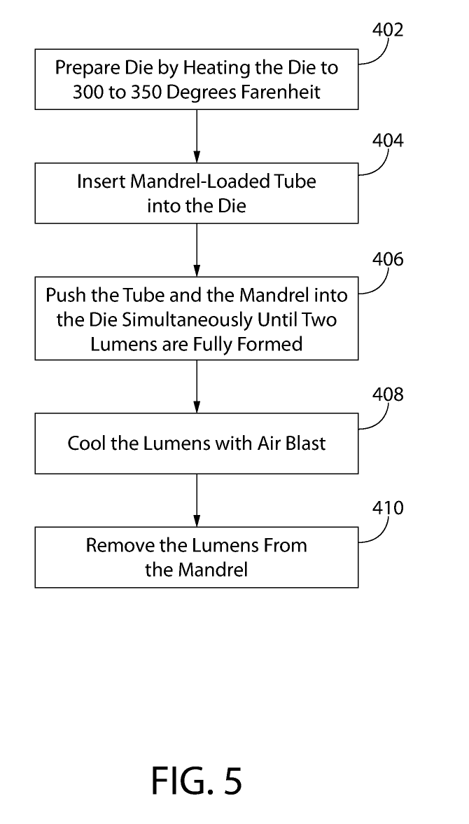

Referring now to FIG. 5 there are shown exemplary method steps for producing split catheters. As discussed above, the die assembly 202 (FIG. 2) comprises a heated die 206 (FIG. 2), wherein the die 206 (FIG. 2) comprises a tubular structure with a circular cross section, the cross section comprising a septum 304 (FIG. 3) disposed at a substantial midsection thereof so as to split the cross section (i.e., the inside) of the die 206 (FIG. 2) into two semicircle sections.

The die 206 (FIG. 2) is configured to receive a pair of mandrels 208A, 208B (FIG. 2) therethrough, wherein the mandrels 208A, 208B (FIG. 2) comprise a D-shaped cross section. More specifically, each of the mandrels 208A, 208B (FIG. 2) is configured to be inserted through a semicircle section 306A, 306B of the die 206 (FIG. 2). Additionally, the mandrels 208A, 208B (FIG. 2) are configured to support a dual lumen 210 (FIG. 2) thereon, wherein the dual lumen 210 (FIG. 2) comprises a circular cross section with a septum disposed in the midsection thereof, defining two open semicircle sections, similar to the die 206 (FIG. 2). The septum 304 (FIG. 3) is configured to split the dual lumen 210 by splitting the dual lumen 210 at the septum of the dual lumen 210.

As indicated in block 402, the die 206 (FIG. 2) is heated to a desired temperature via a heating unit 306. In a preferred embodiment, the die 206 (FIG. 2) is heated to 300° F. to 350° F., via, for example, a heating unit 306 (FIG. 4) utilizing radio frequency technology. As indicated in block 404, the mandrels 208A, 208B (FIG. 2) are positioned within the semicircle sections 306A, 306B of the dual lumen 210 (FIG. 2) (e.g., having an inside diameter of 0.060″×0.130″ according to one embodiment) and then the dual lumen 210 (FIG. 2), while loaded on the mandrels 208A, 208B (FIG. 2), are inserted into the die 206 (FIG. 2). It is noted that the mandrels 208A, 208B (FIG. 2) help prevent the dual lumen 210 (FIG. 2) from collapsing when heat is applied.

As indicated in block 406, the dual lumen 210 (FIG. 2) and the mandrels 208A, 208B (FIG. 2) are pushed, for example, via a block 216 (FIG. 2), into the die 206 (FIG. 2) until the dual lumen 210 (FIG. 2) passes through the septum 304 (FIG. 2) within the die 206 (FIG. 2) and two lumens are fully formed. The combination of properly guiding the dual lumen 210 and the heat creates a smooth finish. In this regard, no additional process is needed to improve the finish of the split lumens. As indicated in block 408, the separated lumens are cooled down with air blast via a cooling unit 308 (FIG. 4). Finally, as indicated in block 410, the lumens are removed from the mandrels 208A, 208B (FIG. 2). In this regard, regular dual lumens 210 (FIG. 2) can be used and the septum of the dual lumen 210 (FIG. 2) do not need to have additional thickness.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

1. A method of manufacturing a split tip catheter assembly having a dual lumen having a proximal end, a distal end, and a septum extending from said proximal end to said distal end, wherein said septum comprises a thickness that is substantially equal to a thickness of said dual lumen, the method comprising the steps of:
providing a heated die comprising a circular cross section with a septum located at a midsection thereof;
loading said dual lumen on a pair of mandrels;
inserting said pair of mandrels loaded with said dual lumen through said heated die;
guiding said dual lumen and said pair of mandrels through said heated die until said septum of said heated die separates said dual lumen into two separate lumens.

2. The method of claim 1, wherein said heated die is heated via a radio frequency heating system.

3. The method of claim 1, further comprising the steps of:
cooling said lumens via air blast; and
removing said two separate lumens from said pair of mandrels.

4. The method of claim 1, wherein said heated die is heated to 300° F. to 350° F.

5. A device for manufacturing a split tip catheter, comprising:
a die comprising a circular cross section with a septum located at a midsection thereof, thereby defining a first semicircular section and a second semicircular section, wherein said first semicircular section is configured to receive a first mandrel therethrough and said second semicircular section is configured to receive a second mandrel therethrough, each of said first mandrel and said second mandrel configured to load a dual lumen thereon, wherein said dual lumen comprises a septum, thereby defining a first lumen and a second lumen;
a heating unit operatively connected to said die for heating said die to a desired temperature range.

6. The device of claim 5, wherein said heating unit comprises a radio frequency heating system. The device of claim 5, wherein said desired temperature range is 300° F. to 350° F.

8. The device of claim 5, further comprising a die holder connected to said die, wherein a distal end of said die holder is connected to a proximal end of said die, further wherein said die holder is configured to receive said first mandrel, said second mandrel, and said dual lumen therethrough.

9. The device of claim 5, further comprising a block, wherein said block is configured to guide said first mandrel, said second mandrel, and said dual lumen through said die in order to split said septum of said dual lumen with said septum of said die when said die is heated to said desired temperature range in order to separate said dual lumen into two separate lumens.

10. The device of claim 5, further comprising a temperature controller operatively connected to said heating unit for monitoring a temperature of said die.

11. The device of claim 5, further comprising a sensor coupled to said die, wherein said sensor comprises a thermometer.

12. A device for manufacturing a split tip catheter, comprising
a die comprising a circular cross section with a septum located at a midsection thereof, thereby defining a first semicircular section and a second semicircular section;
a heating unit operatively connected to said die for heating said die to a desired temperature range;
a temperature controller operatively connected to said heating unit for monitoring a temperature of said die.

13. The device of claim 12, further comprising a sensor coupled to said die and said temperature controller, wherein said sensor comprises a thermometer.

14. The device of claim 12, further comprising a processor operatively connected to said temperature controller for controlling said heating unit, wherein said processor is configured to instruct said temperature controller to lower or raise said temperature of said die if said temperature controller determines that said temperature of said die is outside of said desired temperature range.

15. The device of claim 12, wherein said heating unit comprises a radio frequency heating system.

16. The device of claim 12, further comprising a cooling unit operatively connected to said die, wherein said cooling unit is configured to deliver air blast.

17. The device of claim 12, wherein said first semicircular section is configured to receive a first mandrel therethrough and said second semicircular section is configured to receive a second mandrel therethrough, each of said first mandrel and said second mandrel configured to load a dual lumen thereon, wherein said dual lumen comprises a septum, thereby defining a first lumen and a second lumen.

18. The device of claim 17, further comprising a block, wherein said block is configured to guide said first mandrel, said second mandrel, and said dual lumen through said die in order to split said septum of said dual lumen with said septum of said die when said die is heated to said desired temperature range in order to separate said dual lumen into two separate lumens.
